(19) United States
(12) Patent Application Publication
(10) Pub. No.: US 2009/0198268 A1
(43) Pub. Date: Aug. 6, 2009

(54) Nasal dilation apparatus

(76) Inventor: Ernest Jerold Case, New Iberia, LA (US)

Correspondence Address: Ted M. Anthony, P.O. Box 3408, Lafayette, LA, 70502, US

(21) Appl. No.: 12/012,455

(22) Filed: Feb. 1, 2008

(51) Int. Cl. A61B 17/24

(52) U.S. Cl. 606/199

(57) ABSTRACT

A device for dilating nasal passageways and improving nasal breathing capacity is disclosed. A curved resilient member having divergent first and second ends is provided. After pinching the ends of said resilient member toward one another, the first and second ends can be positioned in respective nostrils of a user's nose. In this position, the ends of said resilient member are outwardly biased against the nasal side walls, with such biasing force being sufficient to dilate the anterior portion of each nasal cavity by an amount sufficient to improve nasal breathing capacity. An adhesive strip is provided to affix the device to outer surface of the septum of a user's nose.

### CROSS REFERENCES TO RELATED APPLICATIONS

None.

### STATEMENTS AS TO RIGHTS TO INVENTIONS MADE UNDER FEDERALLY SPONSORED RESEARCH AND DEVELOPMENT

NONE

### BACKGROUND OF THE INVENTION

1. Field of the Invention

The present invention relates to an apparatus for improving nasal breathing capacity. More particularly, the present invention pertains to an inexpensive disposable apparatus for dilating nasal passageways in order to open such passageways and permit improved nasal breathing capacity.

2. Brief Description of the Prior Art

The human nose generally comprises two nasal passageways divided by a thin wall, known as a septum. The internal surfaces of such nasal passageways are typically covered with respiratory mucous membranes. Impurities such as dust, bacteria, and chemical particles inhaled into the nose from the surrounding environment are trapped by the mucus present on the mucus membranes. Such impurities are ultimately carried to the throat and swallowed.

Nasal passageways can become collapsed or constricted, especially during sleep. Such collapsed or constricted nasal passageways can frequently result in a number of different health problems including, but not necessarily limited to, respiratory ailments.

One common and serious respiratory ailment that can be caused and/or exacerbated by collapsed or constricted nasal passageways is sleep apnea, which can be characterized by extended pauses in the breathing process during sleep. Sleep apnea episodes can each last long enough for one or more breaths to be missed, and can occur repeatedly throughout sleep.

Snoring is another common problem associated with collapsed or constricted nasal passageways. Snoring typically results from the vibration of respiratory structures (such as the uvula and/or soft palate) due to obstructed air movement during sleep. The resulting audible sounds may be soft or loud, and are frequently very unpleasant. Such sounds can be a nuisance to persons within hearing distance, and can negatively impact the quality of sleep of the snoring person.

A number of different nasal dilation devices have been designed to open nasal passageways in an effort to combat these and other respiratory ailments. Unfortunately, such nasal dilators, which include both external and internal devices, have proven to be lacking in a number of different respects.

One common external nasal dilation device comprises a resilient adhesive strip that can be worn on the exterior surface of a user's nose near the bridge of said nose. The resilient adhesive strip acts to lift and spread the walls of the nose, thereby opening nasal passages in order to improve breathing capacity. However, such external dilation devices are very noticeable to others, and often cause discomfort or damage to the skin and soft facial tissues of the user. Further, external placement of such devices exposes them to a variety of forces that can prematurely cause dislodgement from the nose (such as, for example, rubbing against pillows and the like when worn during sleep).

Internal nasal dilation devices also act to spread the walls of the nose, thereby opening nasal passages to improve breathing capacity. Such existing internal nasal dilation devices can be inserted within the nostrils of a nose, and are typically held in place by compressive forces that are sufficient to hold the devices stationary. However, due to the smoothness and lubrication of the mucous membranes lining the inner surfaces of the nose, such existing nasal dilation devices have a tendency to slide out of a wearer's nose, especially when such devices are being worn during sleep.

Helical coils have been developed that can be inserted into a user's nostrils. Such nostrils contact both the sidewalls of the nasal cavities, and the septum. Such coil devices can significantly disrupt air-flow through the anterior part of each nasal cavity. Such devices cannot be maintained in the nose without a high degree of comfort for the user.

Thus, there is a need for an improved device that can dilate nasal passages to facilitate improved breathing function. Such improved device should be comfortable, effective, and sufficiently unobtrusive for every day use. The device should remain in position, even when it is worn during sleep. Further, the device should be relatively inconspicuous, such that it is not overly noticeable while being used.

### SUMMARY OF THE PRESENT INVENTION

The nasal dilation apparatus of the present invention can be beneficially (partially) inserted within the nostrils of a user's nose to spread open nasal passageways and improve nasal breathing capacity. In the preferred embodiment, the apparatus of the present invention comprises a lightweight, resilient and flexible body member having two ends. Said body member generally forms a “V-shape,” but with a rounded curvature or arc at the mid-point. In the preferred embodiment, the ends of said body member gently flare outwardly relative to said mid-point.

Rounded bulb-like end members are disposed at said first and second ends of said body member; in the preferred embodiment, said rounded end members are larger in size than said body member. Additionally, in the preferred embodiment, an adhesive strip is disposed on said body member near the mid-point (that is, the half-way point) between said first and second ends of said body member.

The apparatus of the present invention should be beneficially constructed of a lightweight, non-toxic and non-abrasive material. Although any number of different materials could potentially be used for this purpose, in the preferred embodiment the present invention is beneficially made of a soft, inert and flexible polymeric material having desired resiliency such as, for example, a synthetic rubber or plastic. For example, a particularly preferred polymeric material for construction of the present invention is a silicone elastomer such as Silastic®), which is frequently used in medical applications because of its compatibility with human tissue. By adjusting physical characteristics such as flexibility, devices having identical dimensions, but with different effectiveness (that is, dilating ability) may be obtained.

In operation, the ends of body member of the present invention is “pinched” inward about its mid-point, such that said first and second ends are generally brought toward one another. The ends of said body member are thereafter inserted in the nostrils of a user, with said first end being received in one nostril and said second end being received in the other nostril. Due to the resiliency of said body member, said first and second ends of said body member are biased outwardly against the inner surfaces of the nasal side walls. Such outward biasing force is sufficient to hold the device in the nose and to dilate the anterior portion of each nasal cavity by an amount sufficient to improve nasal breathing capacity.

In the preferred embodiment, the adhesive strip situated on said body member is positioned against, and affixed to, the lower outer surface of the septum (located near the base of the nose between the nostrils). Said adhesive strip helps to secure the apparatus of the present invention within a user's nose. Even though the first and second ends of said body member are disposed against the relatively slick mucous membranes along the inner surfaces of the nasal passageways, said adhesive strip holds the apparatus securely in place and prevents the apparatus from inadvertently sliding out of the user's nose. Said adhesive will beneficially secure said apparatus in a user's nose, without irritating the skin of the user.

When the apparatus of the present invention is inserted within a user's nose, said rounded end members are beneficially in direct contact with the inner surfaces of said user's nasal passages. Because said rounded end members are larger than the body member and rounded, user comfort is increased. Further, because said rounded end members are larger than the body member, the points of contact with the internal surfaces of the nasal side walls, and the resulting frictional contact between the rounded end members and said internal surfaces, is increased.

Large variations in physical characteristics exist between the noses of different individuals. As such, the size of the components of the present invention can be easily altered to beneficially fit particular users. Similarly, the apparatus of the present invention can be beneficially sized differently to fit men, women and/or children.

A device of the type of the present invention, suitable for dilating the anterior portion of each nasal cavity of an individual, may be used to improve nasal breathing capacity. By way of illustration, but not limitation, the device of the present invention is especially useful for asthma sufferers and habitual snorers.

### BRIEF DESCRIPTION OF THE DRAWINGS

The foregoing summary, as well as the following detailed description of the preferred embodiments, is better understood when read in conjunction with the appended drawings. For the purpose of illustrating the invention, the drawings show certain preferred embodiments. It is understood, however, that the invention is not limited to the specific methods and devices disclosed.

FIG. 1 depicts a front perspective view of the nasal dilation apparatus of the present invention installed in a user's nose.

FIG. 2 depicts a side perspective view of the nasal dilation apparatus of the present invention in a partially bent configuration.

FIG. 3 depicts a side view of the nasal dilation apparatus of the present invention in a partially bent configuration.

### DETAILED DESCRIPTION OF A PREFERRED EMBODIMENT OF THE INVENTION

Referring to the drawings, FIG. 3 depicts a side view of the nasal dilation apparatus of the present invention. In the preferred embodiment, the apparatus of the present invention comprises an elongate and resilient body member 6 having a length, and first and second ends. Rounded end members 4 are disposed at said first and second ends of said body member 6. In the preferred embodiment, said rounded end members 4 are larger in size than rest of body member 6.

Body member 6 substantially forms a “V-shape,” wherein end members 4 gently flare outward relative to the mid-point (that is, the half-way point between the first and second ends) of body member 6 and diverge from one another. However, in the preferred embodiment, instead of a sharp acute angle at its mid-point (i.e., the vertex of the v-shape), said body member 6 has a rounded curvature or arc at said mid-point.

In the preferred embodiment, adhesive device 8 is disposed on the inner surface of body member 6 near the mid-point of said body member 6. Referring to FIG. 3, adhesive device 8 is located within the inner surface of the curved arc formed at the mid-point of body member 6. In the preferred embodiment, said adhesive device 8 comprises adhesive strip 12, and peel-away cover 10. Peel-away cover 10 protects adhesive strip 12 from unwanted contact or removal of adhesive from said adhesive strip 12, and can be easily removed prior to use of the present invention.

FIG. 2 depicts a side perspective view of the nasal dilation apparatus 2 of the present invention in a partially collapsed configuration. In operation, body member 6 of the present invention is “pinched” inward about its mid-point, such that said first and second end members 4 are generally brought toward one another. Due to the spring-like resilient characteristics of body member 6, when forced toward each other, end members 4 exert an opposing force away from one another.

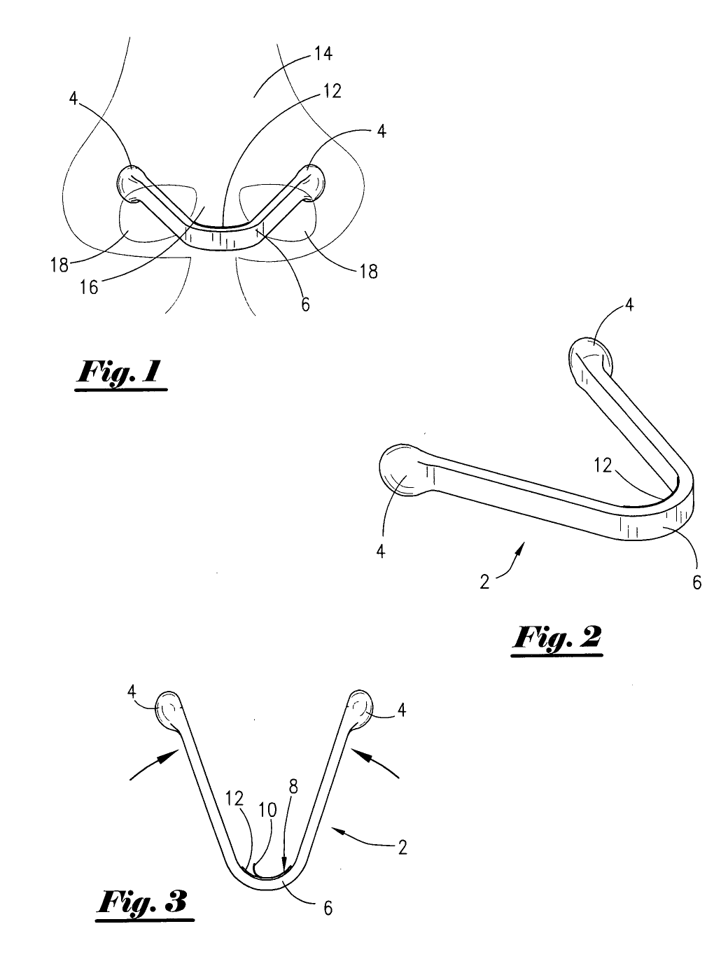

FIG. 1 depicts a front perspective view of the nasal dilation apparatus 2 of the present invention installed in a user's nose 14. In operations, rounded end members 4 of body member 6 are pinched toward one another. Thereafter, said rounded end members 4 are inserted into nostrils 18 of nose 14, with one rounded end member being received in one nostril, and the other rounded end member being received in the other nostril. Due to the resiliency of body member 6, said end members 4 of said body member 6 are biased outwardly against the inner surfaces of the nasal side walls of nose 14. Such outwardly biasing force is sufficient to temporarily secure apparatus 2 within nose 14 and to dilate the anterior portion of each nasal cavity of nose 14 by an amount sufficient to improve nasal breathing.

As set forth above, the apparatus of the present invention should be beneficially constructed of a lightweight, non-toxic and non-abrasive material. Although any number of different materials could potentially be used for this purpose, in the preferred embodiment body member 6 and rounded end members 4 of the present invention are beneficially made of a soft, inert and flexible polymeric material having desired resiliency such as, for example, a synthetic rubber or plastic. By way of illustration, but not limitation, a particularly preferred polymeric material for construction of the present invention is a silicone elastomer such as Silastic®. By adjusting physical characteristics such as flexibility, devices of identical dimensions, but with different effectiveness (that is, dilating ability) may be obtained.

In the preferred embodiment, peel-away cover 10 can be removed from adhesive strip 12 situated on body member 6. Adhesive strip 12 is then positioned against, and affixed to, the outer surface of septum 16 (located near the base of nose 14, between nostrils 18). Said adhesive strip 12 helps to secure the apparatus of the present invention within nose 14. Even though the first and second end members 4 of body member 6 are disposed against the relatively slick mucous membranes lining the inner surfaces of the nasal passageways of nose 14, adhesive strip 12 holds the apparatus securely in place and prevents the apparatus from inadvertently sliding out of nose 14. In the preferred embodiment, adhesive used on adhesive strip 12 can be easily removed, and will not irritate the tissues of nose 14 (including the skin on septum 16).

When the apparatus of the present invention is inserted within nose 14, rounded end members 4 are beneficially in direct contact with the inner surfaces of a user's nasal passages. Because said members 4 are rounded and non-abrasive, user comfort is increased. Further, because end members 4 are larger than body member 6, the area of engagement with the internal surfaces of the nasal side wall, and the resulting frictional contact between such surfaces, is increased.

Large variations in physical characteristics exist between the noses of different individuals. As such, the size of the components of the present invention can be easily altered to beneficially fit a particular user. Similarly, the apparatus of the present invention can be sized differently to fit men, women and/or children.

The above-described invention has a number of particular features that should preferably be employed in combination, although each is useful separately without departure from the scope of the invention. While the preferred embodiment of the present invention is shown and described herein, it will be understood that the invention may be embodied otherwise than herein specifically illustrated or described, and that certain changes in form and arrangement of parts and the specific manner of practicing the invention may be made within the underlying idea or principles of the invention.

What is claimed:

1. An apparatus for dilating nasal passageways comprising:
a. an elongate resilient body member having a first end, a second end and a curved section at the mid-point of said body member, wherein said first and second ends diverge from each other; and
b. adhesive affixed to the inner surface of said curved section of said body member.

2. The apparatus of claim 1 further comprising a peelable cover disposed on said adhesive.

3. The apparatus of claim 1, wherein said apparatus is constructed of at least one non-toxic polymeric material.

4. An apparatus for dilating nasal passageways comprising:
a. a substantially v-shaped and resilient body member having a first end and a second end, wherein the mid-point of said body member defines a curved section;
b. a rounded member disposed at said first end;
c. a rounded member disposed at said second end; and
d. adhesive affixed to the inner surface of said curved section of said body member.

5. The apparatus of claim 1 further comprising a peelable cover disposed on said adhesive.

6. The apparatus of claim 1, wherein said apparatus is constructed of at least one non-toxic polymeric material.

7. An apparatus for dilating nasal passageways comprising:
a. an elongate resilient body member having a first end, a second end and a curved section at the mid-point of said body member, wherein said first and second ends diverge from each other;
b. a rounded member disposed at said first end;
c. a rounded member disposed at said second end; and
d. adhesive affixed to the inner surface of said curved section of said body member.

8. The apparatus of claim 7 further comprising a peelable cover disposed on said adhesive.

9. The apparatus of claim 7, wherein said apparatus is constructed of at least one non-toxic polymeric material.
